(19) United States
(12) Patent Application Publication
(10) Pub. No.: US 2005/0177920 A1
(43) Pub. Date: Aug. 18, 2005

(54) Garment for supporting and shaping the mid-section of a wearer

(76) Inventor: William T. Wilkinson, Salem, NJ (US)

Correspondence Address: Connolly Bove Lodge & Hutz LLP, P.O. Box 2207, Wilmington, DE, 19899-2207, US

(21) Appl. No.: 10/781,406

(22) Filed: Feb. 18, 2004

(51) Int. Cl. A61F 5/02

(52) U.S. Cl. 2/69

(57) ABSTRACT

A garment for supporting and shaping the mid-section of a wearer includes an elastic waistband having at least one tensioning system on its abdominal section. The tensioning system incorporates various loops or rings and straps which can be adjustably manipulated in order to control the tension applied at the abdominal section.

### BACKGROUND OF THE INVENTION

Various types of garments are available for promoting health and appearance benefits to the wearer. One type of garment is an exercise garment which is intended to create a workout during use of the garment. Garments also exist such as girdles and various support garments which are intended to enhance the appearance of a wearer. Such garments traditionally do little to shape or support the mid-section. Support of the low back and abdominal area is very important particularly during exercise. Having a more narrow and shapely waist/hips and flatter abdomen are very important in terms of appearance.

### SUMMARY OF THE INVENTION

An object of this invention is to provide a garment which supports and shapes the mid-section of the wearer and particularly the abdomen.

A further object of this invention is to provide such a garment which has the ability to vary the tension in the garment in accordance with the particular needs and desires of the wearer.

In accordance with this invention the garment includes a torso section having an elastic waistband located just below the wearer's rib cage and just below the wearer's belly button. A tensioning system is provided on the abdominal section of the waistband for adjusting the tension of the abdominal section. The tensioning system in general utilizes a pair of anchor members anchored to the abdominal section and transversely spaced from each other. The tensioning system also includes a direction changing member on the abdominal section spaced from at least one of the anchor members. A length control member extends to and bends around the direction changing member in a reverse direction toward itself so as to shorten the distance between the anchor members and thereby increase the tension at the abdominal section. The tensioning system also includes locking structure for holding the length control member in its bent and reversed tension creating condition.

In a preferred practice of the invention the anchor members, the direction changing member and the length control member are a system of loops and straps. For example, in one practice of the invention a strap is provided on each side of the abdominal section. One end of the strap is anchored to the abdominal section to function as one of the anchor members. A loop or ring is fastened to another portion of the abdominal section and functions as the other anchor member. The strap itself is the length control member and with its free end extending around the loop so as to then be folded against itself. The portion of the loop around which the strap extends is the direction changing member. Various fastening structures such as hook and loop fasteners may be provided on the strap to maintain the strap in its bent and tension creating condition.

The invention may also be practiced where a pair of loops are mounted to the abdominal sections spaced from each other to comprise the two anchor members. A strap would be utilized as the length control member and could be threaded around both rings or loops with the two free ends of the strap then secured together. The portions of the rings around which the strap is bent would be the direction changing members and the strap could include fasteners to hold the strap in its tensioned condition.

Other arrangements of loops and straps could also be used such as having a pair of straps wherein one end of each strap is secured to the abdominal section to comprise the anchor members. A loop or ring could be secured to the free end of one of the straps and the free end of the other strap would pass through and be bent around the loop and secured to itself to provide for the adjustable tensioning.

### THE DRAWINGS

FIG. 1 is a front elevational view of a garment in the form of pants incorporating the body support and shaping system of this invention;

FIG. 2 is a top plan view of the body support and shaping system shown in FIG. 1;

FIG. 3 is a fragmental front elevational view showing a modified form of body support and shaping system in accordance with this invention;

FIG. 4 is a rear elevational view of a body support and shaping systems of this invention showing the inclusion of a back support pad;

FIGS. 5-6 are front elevational views of yet other forms of body support and shaping system mounted on a shirt;

FIG. 7 is a front elevational view of a portion of a garment in accordance with this invention;

FIGS. 8-9 are front and side elevational views of yet another practice of this invention;

FIG. 10 is a front elevational view of still yet another practice of this invention;

FIG. 11 is a rear elevational view of a garment in accordance with this invention;

FIG. 12 is a front elevational view in accordance with another aspect of this invention; and

FIG. 13 is a front elevational view of a garment including a pants and a shirt which may be connected together wherein the pants includes a body support and shaping system in accordance with this invention.

### DETAILED DESCRIPTION

The present invention relates to a body support and shaping system particularly for supporting and shaping the mid-section such as the abdomen, waist, low back and/or hips. The system could be incorporated in a garment which functions as overwear, underwear or the sole garment being worn. The system could be incorporated in various types of clothing such as 1) pants, 2) shirt/top/jersey, 3) vest, 4) jacket, 5) sweat or athletic pants, 6) sweat or athletic jersey/top, 7) shorts, 8) one-piece body suit, 9) ski suit, and 10) underwear.

Preferably the system would be incorporated at the upper portion of pants or the lower portion of a top of shirt. One of the features of the invention is the provision of an adjustable or non-adjustable wide elastic or non-elastic band which would be located around the waist. Preferably, the top edge of the band would be under the rib cage while the bottom edge of the band would be generally near the belly button.

Preferably, the elastic waistband is adjustable in tension by an adjustable tensioning system mounted to the abdominal section of the waistband. Preferably, the tensioning system incorporates at least one ring or loop and at least one strap. The various rings and straps may be arranged so as to result in at least one pair of anchor members transversely spaced from each other on the abdominal section. The system also includes at least one direction changing member spaced from at least one of the anchor members. A length control member which could be a strap or spring or any other elongated member extends to and is bendable around the direction changing member in a reverse direction toward itself and preferably into contact with itself to shorten the distance between the anchor members thereby increasing the tension at the abdominal section. Locking structure is also provided for holding the length control member in its bent and reversed tension creating condition. Such locking structure could be any suitable type of fastener and preferably is hook and loop structure.

It is preferable to provide the tensioning system in such a manner that it uniformly creates tension on both sides of the front longitudinal centerline of the waistband or abdominal section. Where two separate systems are used with one on each side it is possible, of course, to have a greater tension being applied from one of the systems than from the other.

One of the preferred features of the invention is the utilization of a wide elastic waistband that can be adjustable, but preferably is not adjustable. The elastic waistband supports and shapes the back, waist and hips. Another feature is an abdominal section which incorporates the tensioning system for adjusting the tension. The abdominal section can be non-adjustable but is preferably adjustable in length. Any suitable combination of components may be used in the tensioning system which achieves the intended results. Preferably, the components comprise a combination of at least one ring or loop and at least one strap. Similarly, the system should include locking structure which is preferably fastening structure to hold the strap or length control member in its bent and tension creating condition. A preferred form of fastening structure is hook and loop elements commercially available as VELCRO®. In the preferred practice of the invention the strap is bent upon itself and secured together. The invention, however, may be broadly practiced where the strap is bent toward itself, but the free end is secured directly to the abdominal section and not necessarily upon the fixed end of the strap.

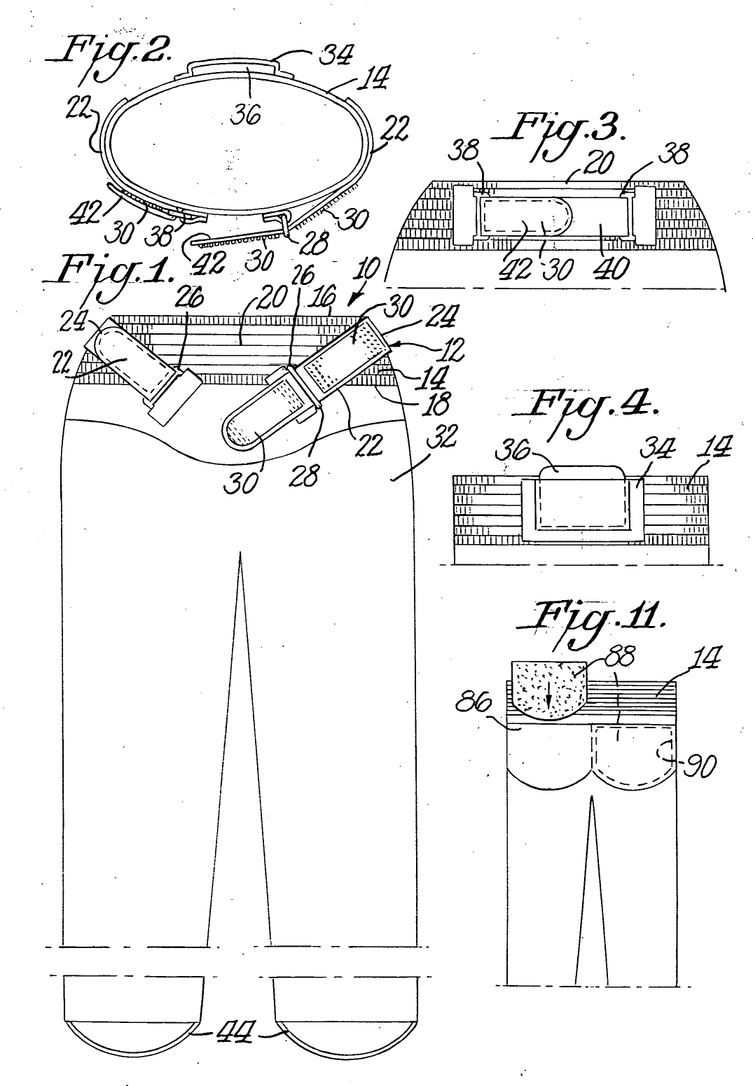

FIGS. 1-2 illustrate one practice of this invention wherein a garment 10 includes a body support and shaping system 12. In the embodiment shown in FIGS. 1-2, the body support and shaping system 12 includes a wide waistband 14 having an upper edge 16 which would be located just below the wearer's rib cage and a lower edge 18 which would be located just below the wearer's belly button. Preferably, the waistband 14 is elastic. The waistband is preferably not adjustable in length or circumference. The invention, however, could be practiced where the waistband could include, for example, segments that could be adjustably connected together to thereby adjust the effective length or circumference of the waistband.

As shown in FIGS. 1-2, the waistband 14 includes an abdominal section 20 which generally extends from one side to the other at the front of the waistband. A tensioning system is provided on each side of the abdominal section 20. In the embodiment illustrated in FIGS. 1-2, each tensioning system includes a strap 22. One end 24 of each strap is permanently mounted or anchored to the abdominal section. In that sense the fixed end may be considered as an anchor member. Spaced inwardly from the first anchor members 24,24 are a pair of loops or rings 26 which are also permanently mounted to the abdominal section 20 on each side of the front longitudinal center line. These fixed rings or loops 26 may be considered as second anchor members. In the embodiment shown in FIGS. 1-2 the free end of each strap 22 passes around its respective ring 26 and is folded in a reverse direction back against itself. FIG. 1, for example, illustrates, in the right-hand portion, one of the straps 22 where it is threaded through the ring 26, while the other strap 22 in the left-hand portion has been bent back against itself. The upper loop portion 28 of the ring 26 functions as a direction changing member since the strap 22 is pivoted or bent around that portion of the loop. The body support and shaping system 12 also includes locking structure which could be any suitable form of fastener such as buttons, clips, tape, etc. A preferred form of fastener is the hook and loop formation 30,30 on opposed surfaces of strap 22 so that the strap 22 is effectively held in the locked tension creating condition once the free end is pressed against the anchored end of the strap.

Thus, the tensioning system 12 shown in FIGS. 1-2 utilizes structure on each of the sides of the waist that includes a strap passing through a ring on each side of the abdominal area. The strap is pulled backward and is fastened to itself through the use of any suitable fastener such as snaps, hooks/loops, buttons, buckles, etc. This creates an adjustable tensioning system that pulls the two anchor members 24,26 toward each other to flatten, shape and support the areas of the low back, the side of the waist, the abdominals and the hips.

While the tensioning system 12 is useful in itself for supporting and shaping the mid-section of the user, the system could also be incorporated in resistance clothing to provide the correct anchor pressure about the waist for the various resistance members that are used to exercise the arms. This would keep the waist from moving from side to side since the straps are pulled forward by the arms for exercise. The inventor herein has numerous patents illustrating various types of resistance clothing which could be used for incorporating the tensioning system of this invention. Examples of such patents are U.S. Pat. Nos. 5,176,600, 5,186,701, 5,306,222, 5,700,231, 5,720,042, 5,842,959, 5,867,826, 5,867,827, 6,047,405 and 6,053,852, all of the details of which are incorporated herein by reference thereto. Various other types of resistance garments which may incorporate the present invention are patents issued wherein the inventor herein is a co-patentee.

The present invention provides a great improvement over the traditional type of approaches taken which do little to shape or support the mid-section. Support of the low back and abdominal area are very important particularly during exercise. The adjustable tensioning aspect of the invention makes the present invention universal in that people of all sizes and shapes can achieve the level of comfort, shape and support desired. Clothing incorporating the present invention might also have medical applications which require adjustable support of the low back and abdomen and help to stabilize the mid-section.

The adjusting system of this invention also provides a marked improvement over traditional waist adjustments such as belts, drawstrings, snaps, buttons, zippers and hooks and loops.

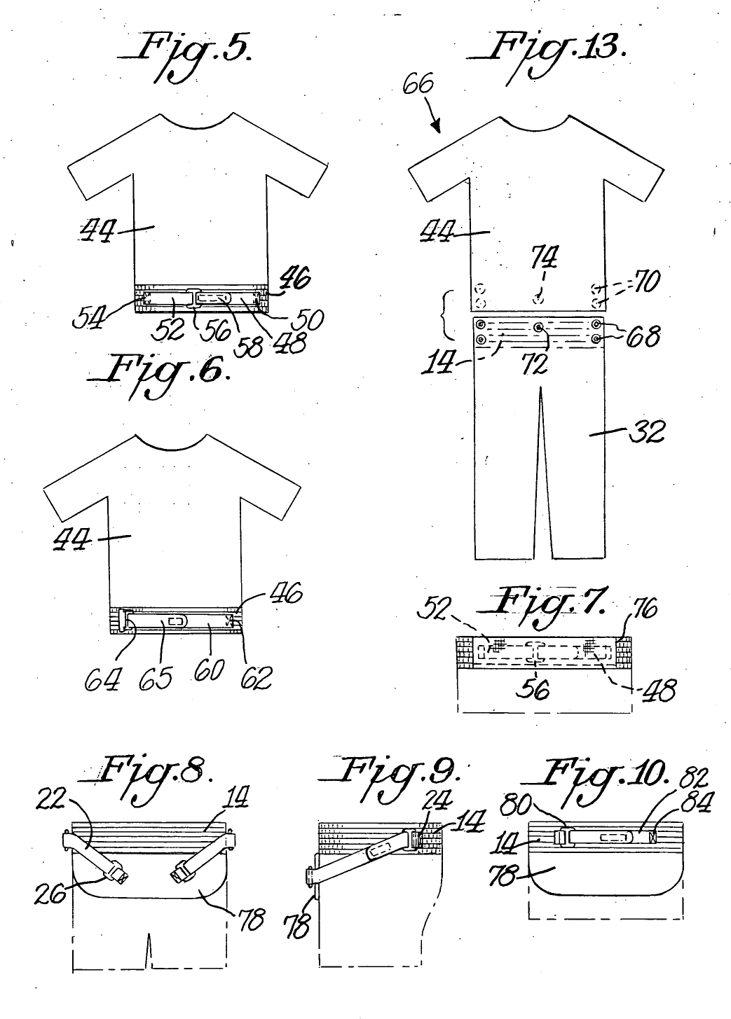

In a preferred embodiment of the invention a wide elastic waistband is provided at the top of the pants as shown in FIGS. 1 and 3 or at the bottom of a shirt or top as shown in FIGS. 5-6. The waistband is preferably of an elastic material and is adjustably tensioned, preferably through the use of one or more straps and preferably through the use of non-elastic straps that are anchored to the sides of the waist and run through a loop anchored on each side of the abdominal region as shown, for example, in FIGS. 1-2. When the user pulls the strap back, the ring or base loop pulls the abdominal section 20 which flattens the abdominal area and gives that area support. The strap itself is preferably attached to itself, although the strap could be attached to adjacent portions of the abdominal section 20.

The invention marks a vast improvement over the drawstring/elastic waist techniques commonly used on most sweat pants or shorts where the intent is simply to keep the pants up but not provide any shaping or support function.

When the invention is applied to a shirt or top the same system would be used, but would be located at or near the bottom of the shirt. Thus, the same effect for narrowing the waist, supporting the low back, shaping the hips and flattening the abdomen would be achieved in a shirt or top just as with the pants.

With conventional shirts or tops the bottom of the shirt or top is usually loose and offers little or no shaping and support to the waist and abdomen areas. The present invention, in contrast, not only provides body support and shaping benefits, but has the added benefit of improving the ability to hold the garment in place at the waist.

The invention could also be practiced utilizing elastic material for the pants section 32 that covers the buttock and thighs. Thus, the system of the invention could be coupled or incorporated into an elastic pants or shorts or compression garment that will additionally provide support and shape for the thighs and buttock while the invention supports and shapes the abdomen.

The invention also can provide improved comfort about the waist and mid-section by incorporating materials that wick away moisture and dispense heat in hot/warm weather conditions. Conversely, the garment could include warm materials, such as flannel to provide comfort in cold weather. Comfort would also be provided by the use of soft cushion-type materials such as memory foam that reduces pressure points. The invention could also incorporate materials that promote weight loss such as heat materials that promote sweating or cool/cold materials to promote heat transfer/calorie burning. An example of this practice would be to sandwich heat pack materials, such as conventional gels known for that purpose, between layers of the fabric forming the garment.

Another variation of the invention is to incorporate additional structure such as a foam pad or insert to provide additional back support which may be adjustable in height or pressure/dimension. FIG. 4, for example, illustrates the utilization of a pocket 34 in the back portion of the waistband 14 in which a back support pad 36 is preferably detachably mounted.

The entire support and shaping system 12 could be removably or detachably mounted to the waist portion of the shirt or pants, but it preferably a permanent part.

Another feature of the invention is that the angle of tension can be varied in accordance with different structural arrangements. FIG. 1, for example, illustrates the pull from the straps 22 to be angular between the anchor members 24,26. As later described FIG. 3 shows the pull to be straight or horizontally in line with the anchor members. Depending on the location of the anchor members or straps, the pull could be arranged at any angle, could be up, down or sideways. Where the support and shaping system is used on pants it is preferable that there should be an upward pull or a sideways pull. This provides an anchor for the pants and helps keep the pants up rather than slipping down. Conversely, for the top it is preferred that the pull should be downwardly or sideways. This provides an anchor for the top and tends to keep the top down rather than slipping up.

FIG. 3 illustrates a further practice of the invention wherein the support and shaping system includes a pair of loops or rings 38 mounted to the abdominal section 20 transversely spaced from each other. A single strap 40 is looped through the rings 38,38 and folded upon itself so that its free ends 42 can be secured together after the desired amount of tension has been applied pulling the rings 38,38 closer together. In this embodiment the two rings would be the anchor members. The portions of the rings around which the strap 40 is bent would be the direction changing members and the strap 40 would be the length control member with the hooks and loops on the inner surface of the free end 40 of the strap 42 and on its abutting strap surface being the locking structure. In this embodiment, the tensioning is in a straight, horizontal direction rather than at an angle.

In the embodiments of FIGS. 1-2 and of FIG. 3 the support and shaping system 12 is incorporated in pants. Such pants may include other features such as stirrups 44 shown in FIG. 1 for size and comfort adjustment.

Other variations include the possibility of having the support pad 36 shown in FIG. 4 being inflatable or non-inflatable and being removable or permanently included in the pocket. Where the pad is permanent the pocket could be completely closed. Where the pad is removable the pocket should be open on at least one edge. For example, where the waistband 14 is made of at least two layers the pocket could be formed by sewing the layers together to form the longitudinal edges of the pocket. The lower edges of the layers would also be secured together, but the upper edges could be slit to form the open end of the pocket for facilitating the insertion and removal of the pad.

Although FIGS. 1-4 illustrate the support and shaping systems to be incorporated at the top of a pants, as previously indicated the system could also be incorporated at the bottom of a shirt. FIG. 5, for example, shows a garment which includes as the torso section a shirt 44 which includes a waistband 46 similar to waistband 14 except that it is located at the bottom of the shirt rather than at the top of the pants. FIG. 5 also illustrates a variation of the support and shaping system that could be used either on a shirt or pants. As shown therein the system includes a base strap 48 anchored at its one end 50. A second strap 52 is anchored at its one end 54. A buckle or ring or loop 56 is secured at the free end of strap 52. In order to provide tensioning of the abdominal section the free end 58 of base strap 48 is inserted through the buckle or loop or ring 56 and then bent upon itself through the use of any suitable fastener such as hooks and loops as previously described. In this embodiment the two anchor members would be the fixed ends 50 and 54 of the straps 48 and 52. The loop 56 would function as the direction changing member and the strap 48 would function as the length control member.

FIG. 6 shows yet another variation of a support and shaping system which is illustrated as being on a shirt 44 located at the abdominal section of the waistband 46. In the version shown in FIG. 6 a base strap 60 is anchored at its one end 62 to the abdominal section spaced from a ring or loop 64 anchored on the other side of the abdominal section. The free end 66 of strap 60 is inserted through the loop or ring 64 and bent back upon itself to be secured to itself through any suitable fasteners as previously described. In this embodiment the fixed end 62 and the loop 64 function as anchor members. The portion of the ring or loop 64 through which the free end 66 of strap 60 is passed functions as the direction changing member and the strap 60 is the length control member.

Preferably, in the various practices of this invention the tensioning is applied essentially symmetrically with regard to the longitudinal center line at the front of the waistband or abdominal section. Thus, where two tensioning systems are used each tensioning system is spaced away from the longitudinal center line the same distance as each other. Where only a single tensioning system is used the anchor members of that tensioning system are preferably spaced from the longitudinal center line the same distance as each other. It is to be understood, however, that a symmetrical location of the tensioning systems or of the anchor members is not absolutely necessary with the practice of this invention. Similarly, it is not necessary that the same amount of tension be applied on each side of the longitudinal center line, although such is preferred.

If desired, the invention could be practiced where, for example, the base strap which passes through a ring is larger and longer than the strap which holds the ring. This would permit the strap holding the ring to be made more narrow for style purposes and at less cost yet engage a larger area when pulled so that it flattens and shapes more of the abdomen and waist.

The invention may also be practiced in its broad aspect wherein the added tension is created at the abdominal section by a tightening mechanism which does not require any changing of direction of a length control member. For example, the arrangement illustrated in FIG. 5 could be modified wherein the tightening mechanism could still include the two spaced anchor members resulting from the ends 50 and 54 of straps 48 and 52 being permanently secured to the abdominal section. One of the straps, such as strap 52 could have on its free end as part of the tightening mechanism a buckle 56 which would be of a structure to engage the free end 58 of the base strap 48 in an adjustable manner by simply inserting the free end 58 through the buckle 56 without reversing the direction of the free end 58. The two anchor members 50,54 would be pulled closer together and would be held in this increased tension created condition in any suitable manner such as by the buckle being engaged with one of a selected number of holes in free end 58 similar to a conventional belt. Other tightening mechanism alternatives could be through the use of different fasteners such as Velcro®, clips, snaps, hooks, etc. which would permit the anchored ends 50,54 to be pulled closer together and then locked in that increased tension created condition.

The various components of the tensioning system could be made less conspicuous by various techniques, such as forming the straps or other components of the same color as the adjoining parts of the garment or with patterns or designs that disguise the components. FIG. 7 shows a further alternative wherein a tunnel 76 is provided to cover the tensioning system during periods of either use or nonuse. The tunnel could be a fabric flap which extends over the straps and other components. Alternatively, the tunnel could be provided adjacent to each of the straps so that the straps could be folded back and inserted into the tunnel during periods of non-use. A further technique would be to mount the tensioning system on the inside of the garment so that it is thereby completely concealed.

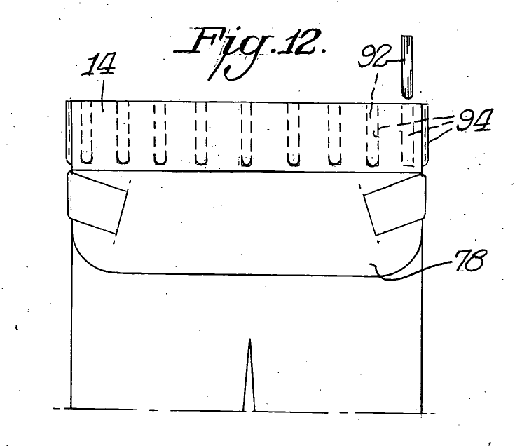

FIGS. 8-9 illustrate a practice of the invention wherein the abdominal section or panel 78 is located below the waistband 14. As shown in FIG. 9 the abdominal panel 78 is provided only at the front of the garment and does not extend completely around the garment. The strap 22 has its anchored end 24 at the side of the waistband and extends to the abdominal panel 78 with the anchored ring or loop 26 being located on the abdominal panel. Tensioning system otherwise functions in the same manner as in FIGS. 1-2. Preferably, Velcro® hooks and loops are used on the straps to provide the desired adjustment.

In a sense, the system works similar to a girdle but is much easier to adjust and more comfortable by being incorporated into a regular garment part such as the pants.

A wider waistband is not adjusted well with traditional drawstrings. One feature of the invention is to provide better adjustment for the waistband. FIG. 10, for example, illustrates waistband adjusting structure which includes a ring 80 on one side of the waistband 14 and a strap 82 on the other side of the waistband. The strap 82 has an anchored end 84 secured to the waistband. The strap 82 passes through the loop or ring 80 and is pulled back upon itself similar to the straps of the tensioning system. The strap can then be fastened in the pulled back position by any of the types of fastening structures used with the tensioning system. The strap and ring arrangement of FIG. 10 thus acts as a belt to provide better control or adjustment of the wide waistband 14. In addition, this arrangement is quick and easy to adjust. Alternatively, one strap could extend completely around the back with a loop on the other strap.

The invention could also be practiced by providing padding or cushioning material permanently sewn to or removably inserted in the garment to shape and protect while cushioning or supporting the buttock. Preferably, the cushioning material would be removable as shown, for example, in FIG. 11. As shown therein, a buttock panel 86 extends downwardly from waistband 14. The buttock panel includes a set of pockets 90 for removably receiving cushioned inserts or foam pads 88 as shown in the lefthand portion of FIG. 11 or the foam pad 88 could be permanently secured as a lining or part of a laminate to the panel 86 as shown in the right-hand portion of FIG. 11. Where the cushioning insert is removable it has the advantage of being able to be washed or otherwise cleaned or to be replaced if necessary. The buttock cushion pads 88 function to shape, to protect and to make the buttock more comfortable by reducing pressure. If desired, instead of having a pair of pads across the buttock a single pad could be used.

FIG. 12 shows a further feature of the invention wherein flexible stays 92 are provided in the waistband 14. Preferably, as illustrated the stays 92 are removable by being insertable into appropriately sized pockets 94. The stays 92 are adjustable in shape so that they can assist in shaping and supporting the areas of the midsection at waistband 14, particularly the hips, low back, waist, and upper abdomen. In this manner they can act as an adjustable corset. As illustrated in FIG. 12 an abdominal panel 78 is also provided below waistband 14. Abdominal panel 78 is preferably made of an elastic material.

Again with reference to FIG. 12 the stays 92 can be removed for replacement to vary the shape and positioning of the stays. The stays are seated in the pockets 94 inside the pants or shorts that are in the various locations around the waist. The stays can extend below the waist.

The invention can be practiced where the portion of the garment having the support and shaping system on its torso at the waist of the torso, is part of a more complete garment. This could be accomplished by having that portion of the garment join one or more pieces of other clothing to create a more complete garment such as a combined top and pants. FIG. 13, for example, illustrates a garment 66 which includes a pants 32 and a shirt 44, either of which could include the support and shaping system. The pants and shirt could be joined together by any suitable means of attachment such as snaps, hooks and hasp, hooks and loops, straps, rings, buckles, buttons, zippers, suspenders, loops. It is preferable that whatever type of attachment is used should not be permanent but should be detachable to permit the garment 60 to be readily put on and removed. The benefits of having the garment formed of two pieces of clothing are that 1) the complete garment is shaped giving a more trim appearance, 2) a better anchoring system is provided keeping the top from pulling up and the pants from sliding down, 3) wrinkles are reduced because the garment is pulled taut giving a sleeker look, 4) the garment pieces are pulled together keeping air out and making the garment warmer, 5) the garment can be more readily stored or hung by hanging only one piece with the other piece attached, and 6) by bringing the garment pieces together a better appearance is achieved making it less revealing.

The preferred embodiment of this invention is a top and pants, such as a sweatsuit joined together by snaps. Snaps at the bottom of the top can be snapped to snaps at the top of the pants. The snaps could be at different levels. For example, FIG. 13 shows the snaps 68 to be in vertical sets for selective attachment with the vertical set of snaps 70. This provides for vertical edge adjustability. The snaps could be at different points horizontally, circumferentially around the pants and shirt to provide a secure front to back and side to side locking and adjustment. Thus, FIG. 7 also illustrates a set of central fasteners 72,74 to add to the multiple locations where the top and pants are secured together.

As is apparent from the previous discussion the general purpose of the invention is to improve the function of clothing. The primary purpose is to shape and/or support and/or cushion the mid-section of the body. Preferably, the invention shapes, supports and cushions the mid-section all in one garment.

A secondary purpose of the invention is to improve sizing and comfort.
1. The garment can be a pant, top or one piece body suit, but is preferably a pant or short, particularly a sweat pant.
2. It can be over wear, regular wear or underwear, but is preferably regular wear.
3. It shapes and supports and cushions one or more of the areas of the midsection: waist, upper abdomen, low back, hips, lower abdomen, buttock, thighs or groin. Preferably, it shapes and supports the waist, hips, upper/lower abdomen and low back areas all in one garment.
4. The invention can be a pant or short that has one or more shaping, supporting and cushioning structures for the midsection.
5. The structures can be permanently built-in, and/or removable, but are preferably permanent.
6. The structures can be adjustable, non-adjustable, or a combination. Preferably the structures are adjustable, or a combination.
7. The structures can be adjustable by any means, but preferably by a mechanism(s) that is done easily and quickly by hand. Adjustable means includes, but are not limited to: snaps, velcro, buttons, laces and eyelets/holes or straps, hooks, clips, flexible/malleable belts, loops/hasps, drawstrings, strays, buckles, rings, post in holes or stays in pockets. Preferable means of adjustment are: straps, laces and eyelets/hooks, loops/rings, velcro, belts/buckles, flexible stays/pockets or drawstrings.
8. The shaping and supporting structure(s) can be elastic or nonelastic, or a combination. Preferably they are elastic or a combination. Preferably the shaping and supporting structures are elastic, while the adjustment structures are nonelastic. This permits freedom of movement and comfort, yet an adjustment that has control and strength.
9. Such garments have many different/unique features when compared to normal/traditional garments/pants: a) a wide and strong elastic waistband that can go above, overtop, or below the belly button, preferably it goes above and/or over the belly button, to support and shape the upper abdomen, hips, waist and/or back areas; b) an abdominal front panel/section to shape and support the mid and lower abdominal area; c) adjustment structure(s), one or more; and d) permanent or removable padding material or cushions, for comfort and shaping, particularly the buttock. Preferably the pad is made of a viscous or “memory foam”, that molds to the buttock and gives superior support, reducing pressure points, when sitting. When standing, it gives the buttock a more full and shapely appearance. The pads/cushions can be removable for washing and sizing.
10. Ordinarily sweat pants have a thin, weak elastic band and drawstrings. Drawstrings a) tend to break, b) shrink and get lost inside the hole/tunnel, c) just keep pants up and cut into/make lines around the waist. A preferred form of the invention has a Velcro® strap/belt and ring at the waist, for quick/easy adjustment, for comfort, sizing, support and shaping, about the waist, without the drawbacks of drawstrings.
11. Another preferred form of the invention combines various adjustment means to a) flatten and shape/support the abdominal region while b) supporting the back, and adjusting the pressure/support about the waist. One preferred system design is where a strap engages each side of a front abdominal panel.

The base of the strap is preferably wider than the strap, so that a larger area of the abdominal panel can be pulled upward. The other end of the strap passes through a loop on the side of the waist, and then is pulled forward and fastened to a Velcro® patch, or other fastener. When pulled forward, the strap pulls the abdominal panel upward, increasing tension, and thus flattening the abdomen at the same time providing more support. Since the strap ring is based on the wide elastic waistband, when the strap is tightened/drawn forward, this also tightens the waist, and puts additional support pressure on the low back, which is beneficial. The pressure, fit shaping, support, can be easily varied or adjusted, simply by detaching the strap on each side, and relaxing or tightening them.

1. A garment for supporting and shaping the mid section of the wearer, said garment comprising a torso section, said torso section having a wide waistband with an upper edge and a lower edge, said waistband adapted to be located with said upper edge below the wearer's rib cage and with said lower edge generally near the wearer's belly button, said waistband including an abdominal section, a tensioning system for adjusting the tensioning of said abdominal section, said tensioning system comprising a first anchor member anchored to said abdominal section, a second anchor member anchored to said abdominal section transversely spaced from said first anchor member, a direction changing member on said abdominal section spaced from at least one of said anchor members, a length control member extending to and bendable around said direction changing member in a reverse direction toward itself in such a manner to shorten the distance between said anchor members and increase the tension at said abdominal section, and locking structure for holding said length control member in its bent and reversed tension creating condition.

2. The garment of claim 1 wherein said tensioning system includes at least one loop and at least one strap, said direction changing member being at least part of said loop, and said length control member being said strap.

3. The garment of claim 2 wherein said waistband is elastic, and said tensioning system being permanently secured to said abdominal section.

4. The garment of claim 3 wherein said strap includes a first end secured to said abdominal section to comprise said first anchor member, said loop being secured to said abdominal section to comprise said second anchor member, and said strap having a free end which is insertable through said loop and bendable to said reversed tension creating condition.

5. The garment of claim 4 wherein said locking structure comprises hook and loop formations on opposed surfaces of said strap.

6. The garment of claim 4 wherein one of said tensioning systems is located on each side of the longitudinal center line of said abdominal section.

7. The garment of claim 6 wherein said first anchor member and said second anchor member are disposed in line with each other at a non-perpendicular and non-zero angle to said longitudinal center line.

8. The garment of claim 7 wherein said torso section is the top of pants, and said angle being in a direction which extends upwardly and outwardly with respect to said longitudinal center line.

9. The garment of claim 7 wherein said torso section is at the lower portion of a top, and said angle extending downwardly and outwardly with respect to said longitudinal center line.

10. The garment of claim 6 wherein said tensioning systems are horizontally aligned with each other.

11. The garment of claim 4 wherein said strap comprises a single strap which extends from its anchored end and is insertable through said loop and is foldable upon itself.

12. The garment of claim 11 wherein said anchor members of said tensioning system are dis-posed on opposite sides of said longitudinal center line.

13. The garment of claim 3 wherein said tensioning system comprises two spaced loops each of which is secured to said abdominal section to comprise said first anchor member and said second anchor member, and a single strap extending from one of said loops to the other of said loops with said strap having a free end which is insertable through one of said loops and is bendable upon itself.

14. The garment of claim 3 wherein said tensioning system includes two straps, each of said straps having one end attached to said abdominal section to comprise said first anchor member and said second anchor member, said loop being attached to the free end of one of said straps, and the free end of the other of said straps being insertable through said loop and bendable upon itself.

15. The garment of claim 3 wherein said tensioning system includes a single strap, one end of said strap being secured to said abdominal section to comprise said first anchor member, said loop being secured to said abdominal section to comprise said second anchor member, and said strap having a free end which is insertable through said loop and bendable upon itself.

16. The garment of claim 1 wherein said garment includes a pants and a top detachably and vertically adjustably connected to each other, and said tensioning system being on one of said pants and said top.

17. The garment of claim 1 wherein said waistband includes a back support at a location opposite said abdominal section.

18. The garment of claim 17 wherein said waistband has a pocket with an open side, and said back support being a pad removably insertable into said pocket.

19. The garment of claim 1 wherein said waistband is made of a material tending to make the user feel cooler.

20. The garment of claim 1 wherein said waistband is made from a material tending to make the user feel warmer.

21. The garment of claim 1 wherein said waistband includes material for promoting weight loss.

22. The garment of claim 1 including a tunnel located near said tensioning system whereby at least a portion of said tensioning system may be concealed in said tunnel.

23. The garment of claim 1 including adjusting structure on said waistband for adjusting the circumference of said waistband.

24. The garment of claim 1 wherein said waistband includes a plurality of spaced vertical pockets, and a plurality of stays removably insertable into said pockets.

25. The garment of claim 1 including a buttocks panel, and at least one cushioning pad in said buttocks panel.

26. A garment for supporting and shaping the mid section of the wearer, said garment comprising a torso section, said torso section having a wide waistband with an upper edge and a lower edge, said waistband adapted to be located with said upper edge below the wearer's rib cage and with said lower edge generally near the wearer's belly button, said waistband including an abdominal section, a tensioning system for adjusting the tensioning of said abdominal section, said tensioning system comprising a first anchor member anchored to said abdominal section, a second anchor member anchored to said abdominal section transversely spaced from said first anchor member, a tightening mechanism on said abdominal section spaced from at least one of said anchor members for selectively shortening the distance between said anchor members to increase the tension at said abdominal section, and said tightening mechanism including locking structure for holding said anchor members in their increased tension creating condition.

27. The garment of claim 26 wherein said tensioning system includes a pair of straps, each of said straps being anchored at one end to said abdominal section to comprise said first anchor member and said second anchor member, and said tightening mechanism including structure on one of said straps for adjustably engaging the other of said straps.

28. The garment of claim 27 wherein said tightening mechanism includes a buckle on the free end of one of said straps for adjustable engagement with the free end of the other of said straps.

29. The garment of claim 26 wherein said abdominal section is made of a material which wicks away moisture.

30. The garment of claim 29 wherein said abdominal section is part of a pant.

31. The garment of claim 26 wherein said abdominal section is made of a material that promotes weight loss.

32. The garment of claim 31 wherein said abdominal section promotes cooling to cause heat transfer/calorie burning.

33. The garment of claim 26 wherein said tensioning system is located on the inside of said garment so as to be concealed.

34. The garment of claim 29 wherein said abdominal section is part of a pant.

35. In a sweat pants having legs and a torso section above the legs, the improvement being in a waist adjusting system to eliminate the need for a drawstring to adjust the effective waist size of said sweat pants, said torso section generally including the abdomen and waist and hips and buttock and thighs to comprise a mid-section of the sweat pants, said adjusting system being located at said mid-section, said adjusting system comprising a direction changing member anchored to said mid-section, an elongated length control member anchored at one end to said mid-section laterally spaced from said direction changing member, said length control member extending to and being bendable around said direction changing member in a reverse direction toward itself to pull against said direction changing member and adjust the distance between said direction changing member and said anchored one end of said length control member to adjust the waist size, and locking structure for holding said length control member in its bent and reversed direction condition.

36. The sweat pants of claim 35 wherein said direction changing member is a loop, said length control member being a strap, and said locking structure being on said strap.

37. The sweat pants of claim 36 wherein strap is bendable on itself with one surface of said strap being disposed against another surface of said strap, and said locking structure being complementary hook/loop formations on said one surface and on said another surface of said strap.
